(19) United States
(12) Patent Application Publication
(10) Pub. No.: US 2026/0295203 A1
(43) Pub. Date: Oct. 1, 2026

(54) CLOSED-LOOP SLEEP MANAGEMENT METHOD, HEAD-WORN CLOSED-LOOP SLEEP MANAGEMENT SYSTEM, AND ELECTRONIC DEVICE

(71) Applicant: University of Science and Technology of China, Hefei (CN)

(72) Inventors: Tingrui PAN, Suzhou (CN); Binyang DING, Suzhou (CN); Liang YANG, Suzhou (CN); Ruojiang WANG, Suzhou (CN); Kairui WU, Suzhou (CN)

(73) Assignee: University of Science and Technology of China, Hefei (CN)

(21) Appl. No.: 19/304,655

(22) Filed: Aug. 20, 2025

(30) Foreign Application Priority Data
Mar. 31, 2025 (CN) 202510386560.3

(51) Int. Cl. A61M 21/02; A61M 21/00; G16H 40/67

(52) U.S. Cl. CPC A61M 21/02; G16H 40/67; A61M 2021/0027; A61M 2021/0044; A61M 2021/0066; A61M 2205/3303; A61M 2205/3606; A61M 2205/50; A61M 2210/0606; A61M 2230/06; A61M 2230/10; A61M 2230/14; A61M 2230/205

(57) ABSTRACT

A closed-loop sleep management method applied to a head-worn closed-loop sleep management device includes: performing sleep monitoring in real time based on an electroencephalogram (EEG) signal, an electrooculogram (EOG) signal, a heart rate (HR), and a blood oxygen saturation (SpO₂); and controlling an intervention component to perform sleep intervention when a sleep monitoring result meets a preset condition. The sleep monitoring includes sleep staging and calculation of a duration of a current sleep stage. The sleep monitoring result includes the current sleep stage and the duration thereof. The method applies a deep learning algorithm to perform sleep staging and calculate the duration of the current sleep stage based on multi-modal signals, enabling real-time and accurate sleep state monitoring. Additionally, the method can achieve effective dynamic sleep intervention based on the sleep monitoring result, achieving closed-loop management from sleep monitoring to sleep intervention.

### CROSS REFERENCE TO THE RELATED APPLICATIONS

This application is based upon and claims priority to Chinese Patent Application No. 202510386560.3, filed on Mar. 31, 2025, the entire contents of which are incorporated herein by reference.

### TECHNICAL FIELD

The present disclosure belongs to the technical field of sleep state monitoring, and in particular relates to a head-worn sleep staging and sleep intervention technique based on multi-modal signals.

### BACKGROUND

Sleep is crucial for human health, and humans normally spend approximately one-third of their lives sleeping. Research indicates that sleep is essential for the cardiovascular system, and chronic sleep deprivation is associated with cardiac diseases such as hypertension, coronary heart disease, and heart failure. Besides, sleep is significant for emotional regulation and mental health, and insufficient sleep can increase the risk of anxiety, depression, and other psychological problems. Insomnia is a common sleep disorder, which is defined as a sleep latency or wake time after sleep onset exceeding 20 minutes (sleep latency refers to the time from when the individual turns off the light and prepares to sleep until sleep onset). Globally, approximately 10-30% of the population, up to 237 million people, is affected by insomnia. Chronic insomnia may impair social or occupational functioning and reduce quality of life in adults. Individuals with severe insomnia may face increased risks of motor vehicle and workplace accidents, as well as mental and cardiovascular diseases.

Based on the characteristics of various physiological signals, sleep stages are classified according to the American Academy of Sleep Medicine (AASM) standard into wake (W) stage, non-rapid eye movement (NREM) stage I (N1), NREM stage II (N2), NREM stage III (N3), and rapid eye movement (REM) stage. The N3 stage is also called a slow-wave sleep stage, typically referring to a deep sleep stage. As a comprehensive sleep monitoring method, polysomnography (PSG) is the gold standard method for assessing sleep quality and diagnosing sleep disorders (including insomnia, apnea, etc.), and serves as the primary reference for device validation. PSG measures scalp electroencephalogram (EEG), electrooculogram (EOG), chin electromyogram (EMG), electrocardiogram (ECG), respiration, leg movement, nasal pressure, blood oxygen saturation (SpO₂), and body position for sleep monitoring and characterizing sleep disorders. The SpO₂ is measured using a PPG sensor and thus also referred to as a PPG signal. The polysomnograph is the gold standard device for sleep monitoring, capable of measuring the above physiological signals and commonly used by physicians for sleep staging. However, PSG requires specialized acquisition systems and must be recorded, scored, and interpreted by professionals, confining it typically to sleep laboratory research and clinical settings while being time-consuming and expensive. Moreover, the multitude of sensors distributed across the human body during overnight dynamic monitoring in sleep laboratories will cause significant discomfort to patients.

To address these issues, numerous studies have developed wearable portable devices capable of sleep monitoring in non-laboratory environments, with some devices also capable of performing sleep staging. First-generation wearable sleep monitoring devices use actigraphy and built-in accelerometers to measure body movements for inferring sleep and wake states, but they cannot accurately distinguish sleep from motionless wakefulness. Since electrophysiological signals critical for sleep staging and disease diagnosis can be detected at the head, second-generation devices target head as a key location to monitor physiological signals such as PPG and EEG, further enhancing sleep monitoring accuracy. As REMs occur during the REM stage, the REM stage can be accurately identified via EOG signals. However, as the EOG electrodes with complex placement and positioning on both sides of the face are prone to compression during sleep in a lateral position, there are few head-worn devices for detecting EOG signals. EEG signals are weak voltage signals at the microvolt (μV) level, with their amplitude and waveform susceptible to the positions of the acquisition electrodes and reference electrodes. Currently, due to different positions of the acquisition electrodes and reference electrodes, many second-generation devices suffer from non-standardization issues. As a result, most acquired signals fail to meet the medical sleep staging scoring standard in amplitude and waveform, rendering them uninterpretable by professional physicians. Therefore, the accuracy of sleep staging algorithms developed on the basis of unknown signal quality cannot be determined. Additionally, current sleep staging algorithms mostly rely on overnight data analysis, making them hard to achieve real-time sleep staging.

Currently, a variety of devices have shown their potential in sleep intervention, such as transcranial magnetic stimulation (TMS) instrument, transcranial electrical stimulation (tES) instrument, 40 Hz light stimulation glasses, forehead cooling devices, and smart headbands integrating EEG monitoring and acoustic feedback. These devices employ different mechanisms of action (e.g., neuromodulation, light therapy, thermoregulation, and acoustical stimulation), all proven to positively impact sleep quality. However, these technologies advance only in a single sleep intervention function.

Therefore, there remains a lack of a portable and wearable integrated device to achieve real-time and accurate sleep monitoring and further effective sleep intervention.

### SUMMARY

To address the above problems, the present disclosure proposes a closed-loop sleep management method, a head-worn closed-loop sleep management system, and an electronic device. The present disclosure can perform sleep staging and calculate a duration of a current sleep stage through a deep learning algorithm based on multi-modal signals, including an electroencephalogram (EEG) signal, an electrooculogram (EOG) signal, a heart rate (HR), and a SpO₂. Therefore, the present disclosure can achieve real-time and accurate sleep state monitoring. Furthermore, the present disclosure can achieve effective dynamic sleep intervention based on a sleep monitoring result, achieving closed-loop management from sleep monitoring to sleep intervention.

The present disclosure adopts the following technical solutions.

A first aspect provides a closed-loop sleep management method, applied to a head-worn closed-loop sleep management device, and including:
performing sleep monitoring in real time based on an electroencephalogram (EEG) signal, an electrooculogram (EOG) signal, a heart rate (HR), and a SpO₂; and
controlling an intervention component to perform sleep intervention when a sleep monitoring result meets a preset condition;
where the sleep monitoring includes sleep staging and calculation of a duration of a current sleep stage; the sleep monitoring result includes the current sleep stage and the duration thereof; and the sleep stage includes a wake (W) stage, an N1 stage, an N2 stage, an N3 stage, and a rapid eye movement (REM) stage.

A second aspect provides a head-worn closed-loop sleep management system, including: a head-worn closed-loop sleep management device and a computer program, where the head-worn closed-loop sleep management device includes a head-worn wearable mechanism, and a signal acquisition component, an internal circuit and an intervention component that are disposed on the head-worn wearable mechanism; the signal acquisition component includes an EOG electrode, an EEG electrode, a reference electrode, and a PPG sensor; the EOG electrode is configured to acquire an EOG signal; the EEG electrode cooperates with the reference electrode to acquire an EEG signal; the PPG sensor is configured to acquire a PPG signal from a human body; the internal circuit is configured to preprocess the signals acquired by the signal acquisition component, and control the intervention component to perform sleep intervention according to a received intervention command; there is one or more computer programs stored in the internal circuit and/or an electronic device communicatively connected to the head-worn closed-loop sleep management device; and the computer program is executed to implement the closed-loop sleep management method according to the first aspect and any optional solution of the present disclosure.

A third aspect provides an electronic device, including a processor, a memory, and a second communication module, where the electronic device is communicatively connected to a head-worn closed-loop sleep management device via the second communication module; and the processor is configured to call a computer program stored in the memory to implement the closed-loop sleep management method according to the first aspect of the present disclosure.

The present disclosure has the following beneficial effects:
(1) In the present disclosure, the head-worn closed-loop sleep management device acquires the EEG signal, the EOG signal, and the PPG signal from the head, and calculates the HR and SpO₂. The present disclosure performs sleep staging based on the EEG signal, the EOG signal, the HR, and the SpO₂, and calculates the duration of the current sleep stage in real time, achieving real-time and accurate sleep state monitoring. Meanwhile, the present disclosure controls the intervention component integrated in the head-worn closed-loop sleep management device to conduct effective sleep intervention (sleep-inducing intervention and deep sleep intervention) based on the sleep monitoring result. Thus, the present disclosure achieves closed-loop management from sleep monitoring to sleep intervention, thereby effectively alleviating insomnia and promoting deep sleep.

(2) In the present disclosure, the sleep staging method based on multi-modal signals (EEG, EOG, HR, SpO₂ signals) has significant advantages in accuracy and real-time performance. The present disclosure can comprehensively reflect the sleep state by integrating multiple physiological signals, significantly improving staging accuracy. Particularly, the present disclosure can capture subtle changes in slow waves on the EEG, heart rate variability (HRV), and SpO₂ levels through multi-modal signals, achieving high-precision recognition of the N3 stage (deep sleep). Therefore, the present disclosure can reduce errors caused by improper electrode placement or noise in a single signal, enhancing the robustness and accuracy of sleep staging. Moreover, the present disclosure enhances real-time monitoring capabilities through multi-dimensional analysis, provides more reliable technical support for sleep health management, and is suitable for scenarios such as wearable devices.

(3) The present disclosure adopts a sleep staging CNN, which extracts local features of signals (such as EEG rhythm changes) through convolutional kernels in sleep staging tasks and utilizes a multi-layer structure to learn sleep pattern features from low-level to high-level. Thus, the present disclosure significantly improves staging accuracy. The present disclosure reduces computational complexity through parameter sharing and sparse connections, making it suitable for processing high-dimensional physiological signal data. The CNN possesses translation invariance, adapts to individual differences, and features high-parallelism convolution operations, enabling high training and inference efficiency, suitable for real-time applications. Additionally, the CNN supports end-to-end learning without manual feature design, simplifying the complex process of traditional sleep staging.

(4) The present disclosure provides multiple optional intervention methods such as opto-acoustic-thermal regulation, and implements sleep intervention using a combination of opto-acoustic-thermal regulation, achieving high organic integration with wearable devices and providing innovative solutions for sleep management. By combining light stimulation at specific frequencies (e.g., 40 Hz light), acoustic feedback (e.g., pink noise or sleep-inducing audio), and forehead thermal regulation feedback, wearable devices can precisely regulate the user's sleep state, such as promoting deep sleep or alleviating insomnia. Opto-acoustic-thermal regulation intervention features non-invasiveness, low power consumption, and high compatibility, making it highly suitable for integration into wearable devices like smart headbands, eye masks, or earphones. This multi-modal intervention approach enhances device portability and user experience, and enables dynamic closed-loop management through real-time sleep data monitoring, providing personalized and adaptive sleep optimization solutions for users, thereby significantly improving sleep quality.

(5) The present disclosure combines a semiconductor cooling device with a temperature sensor to achieve closed-loop control of core body temperature at the forehead, and uses precise forehead temperature adjustment to intervene in sleep. By monitoring the forehead temperature in real time, the system automatically adjusts the cooling intensity according to a set safety threshold, ensuring temperature changes remain within safe limits to avoid adverse reactions, thereby alleviating insomnia and enhancing sleep quality. Compared to traditional water-cooling circulation methods, this technology offers higher precision, faster response speed, and better wearability.

(6) The present disclosure optimizes the placement of reference electrodes. The reference electrodes are arranged on the inner sides of the straps of the eye mask and located posterior to junctions of auricles with cheeks (overlying the mastoid processes) to provide reference potential signals when the eye mask is worn. The potential signals acquired by the left and right EEG electrodes located on the forehead and the potential differences of the reference signals acquired by the left and right reference electrodes form two EEG signals. Meanwhile, the reference electrode placement site features thin skin and thick bone, and is distant from the cerebral cortex, making it less susceptible to interference from factors such as sweat, body movement, and brain activity. Therefore, the reference electrodes can provide stable reference signals.

(7) The present disclosure optimizes the placement of the EEG and EOG electrodes located on the head and face. The present disclosure optimizes the electrode placement based on the forehead length and width, intercanthal distance, and eye width of Asian adults. Therefore, the electrode placement complies with international PSG wearing standards to ensure high consistency and interpretability of signal characteristics compared to clinical-grade devices.

(8) In the present disclosure, the loop side and hook side of the flexible hook-and-loop fastener work together to achieve flexible adjustment and precise positioning of electrode placement, accommodating deviations caused by differences in human facial structures. During initial wear, users can accurately adjust electrode positions according to their facial structural characteristics, avoiding readjustments during subsequent wear.

(9) The present disclosure designs the head-worn closed-loop sleep management device with a flexible structure, enhancing wearing comfort and minimizing sleep disruption through the use of a textile, a flexible printed circuit (FPC) board, and flexible stretchable electrodes and wires, thereby further improving sleep staging accuracy.

### BRIEF DESCRIPTION OF THE DRAWINGS

FIG. 1 is a schematic structural diagram of a smart eye mask;

FIG. 2 is an exploded view of the smart eye mask;

FIG. 3A shows a first schematic diagram of the smart eye mask being worn, and FIG. 3B shows a second schematic diagram of the smart eye mask being worn;

FIG. 4A shows a schematic structural diagram of an electrode of the smart eye mask, and FIG. 4B shows a schematic structural diagram of a connecting wire of the smart eye mask;

FIG. 5 is a schematic connection diagram of an electrophysiological monitoring electrode;

FIG. 6 is a schematic diagram of PPG measured within a 5-second time window;

FIG. 7 is a flowchart of SpO₂ calculation;

FIG. 8 is a schematic diagram of circuit connections of a smart eye mask system;

FIG. 9 is a flowchart of a real-time sleep staging algorithm;

FIG. 10 is a flowchart of sleep intervention; and

FIG. 11 is a flowchart of closed-loop sleep management.

### DETAILED DESCRIPTION OF THE EMBODIMENTS

The technical solutions of the present disclosure will be clearly and completely described below in conjunction with specific embodiments and drawings of the present disclosure. Obviously, the described embodiments are only a part, rather than all of the embodiments of the present disclosure. All other embodiments obtained by those skilled in the art based on the embodiments of the present disclosure without creative efforts should fall within the protection scope of the present disclosure.

In the description of the present disclosure, orientation or position relationships indicated by terms such as “upper”, “lower”, “left”, “right”, “front”, “rear”, “inside”, “outside”, “top”, and “bottom” are based on the drawings. These terms are merely intended to facilitate description of the present disclosure and simplify the description, rather than to indicate or imply that the mentioned device or element must have a specific orientation and be constructed and operated in a specific orientation. Therefore, these terms should not be construed as a limitation to the present disclosure. Terms such as “first” and “second” are intended to distinguish between similar objects, rather than to indicate specific order or importance. In addition, the terms “including” and “having” and any variations thereof are intended to cover non-exclusive inclusions that may include other units not clearly listed or inherent to these products or devices.

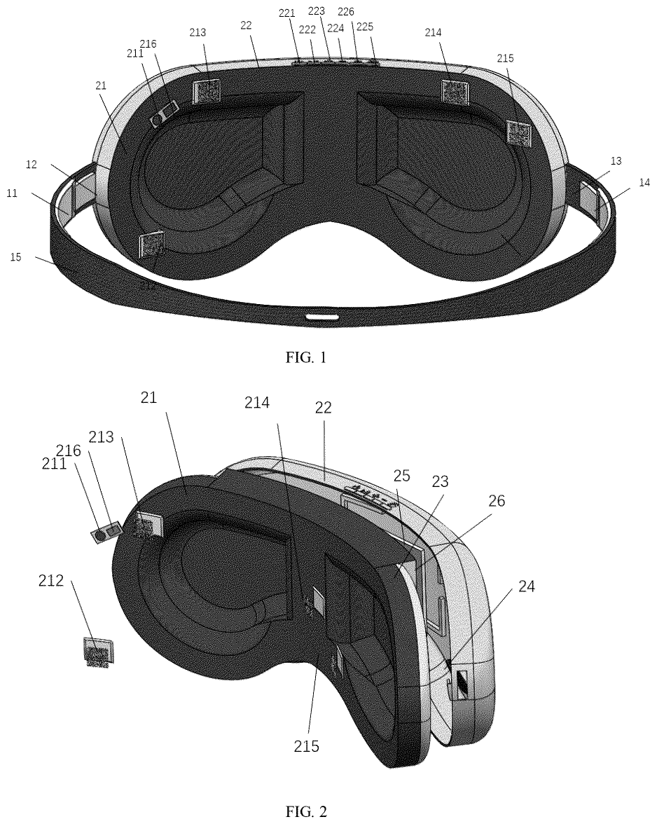

As shown in FIG. 1 and FIG. 2, Embodiment 1 of the present disclosure provides a wearable non-intrusive smart eye mask (referred to as “smart eye mask”), which mainly includes eye mask body 2 and eye mask strap 1 in appearance. The eye mask body 2 mainly includes eye mask liner 21, eye mask shell 22, and support sponge 23. The eye mask body is internally provided with flexible low-voltage light-emitting diode (LED) light strip (referred to as “LED light strip”) 24, internal circuit 25, semiconductor cooling module 26, and a power supply module (not shown in the figure), as well as PPG sensor 27, temperature sensor 28, and electrophysiological monitoring electrodes disposed on the eye mask liner 21.

The eye mask strap 1 mainly includes elastic band 15. Two ends of the elastic band 15 are fixedly connected to the eye mask shell 22. The elastic band 15 is provided with reference electrodes (left reference electrode 11 and right reference electrode 14) and bone conduction earphones (left bone conduction earphone 12 and right bone conduction earphone 13). The eye mask shell 22 is located on an outermost side (a side away from a skin when the eye mask is worn) of the eye mask body 2. The eye mask liner 21 adheres to the support sponge 23, and its edge may be stitched to an edge opening of the support sponge 23 with a stitch to form an integrated structure called the eye mask liner. The eye mask liner 21 and the eye mask shell 22 may be connected by snap-fitting, bonding, insertion, etc. When the eye mask is worn, the eye mask liner 21 closely adheres to a facial skin and is located on an innermost side of the eye mask body.

The eye mask liner 21, the eye mask shell 22, and the eye mask strap 1 collectively constitute a wearable mechanism for carrying components such as the LED light strip 24, the internal circuit 25, the PPG sensor 27, the temperature sensor 28, the reference electrodes, the electrophysiological monitoring electrodes, the semiconductor cooling module 26, the bone conduction earphones, and the power supply module. In terms of material selection, the eye mask liner 21 is preferably made of a medical non-woven fabric, the eye mask shell 22 may be made of a material with good thermal conductivity such as carbon fiber, and the support sponge 23 is preferably made of HKGK high-density sponge.

The electrophysiological monitoring electrodes are disposed on an inner side of the eye mask liner 21 (a skin-facing side when the eye mask is worn), and mainly include left EOG electrode (also referred to as “LEOG electrode”) 212, right EOG electrode (also referred to as “REOG electrode”) 215, left EEG electrode (also referred to as “EEG-FP1 electrode” or “FP1 electrode”) 213, and right EEG electrode (also referred to as “EEG-FP2 electrode” or “FP2 electrode”) 214. The LEOG electrode 212 is located at a lower left side of the eye mask liner 21, i.e. a lower left side of the PPG sensor 27, and adheres to a point 1 cm below an outer canthus of a left eye when the eye mask is worn. The EEG-FP1 electrode 213 is located at an upper left side of the eye mask liner, and adheres to a left forehead when the eye mask is worn. The EEG-FP2 electrode 214 is located at an upper right side of the eye mask liner, and adheres to a right forehead when the eye mask is worn. The REOG electrode 215 is located at an upper right side of the eye mask liner 21, and adheres to a point 1 cm above an outer canthus of a right eye when the eye mask is worn. The EEG-FP1 electrode 213 and the EEG-FP2 electrode 214 are symmetrically distributed about a central axis of the eye mask. A potential difference between the LEOG electrode 212 and the REOG electrode 215 forms an EOG signal. A potential difference between the EEG-FP1 electrode 213 and the left reference electrode 11 forms one EEG signal, and a potential difference between the EEG-FP2 electrode 214 and the right reference electrode 14 forms the other EEG signal.

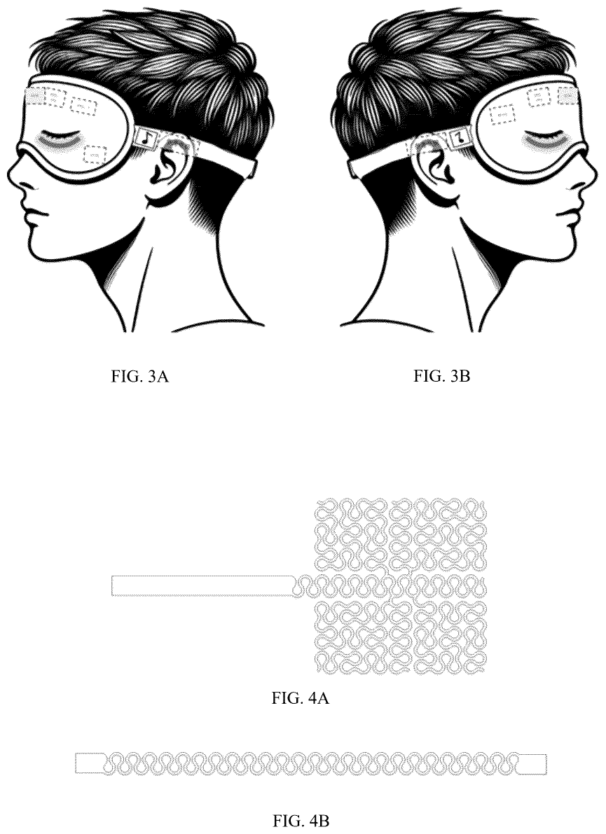

Among the reference electrodes, the left reference electrode 11 and the left bone conduction earphone 12 are disposed on a left side of the elastic band 15, and the right reference electrode 14 and the right bone conduction earphone 13 are disposed on a right side of the elastic band 15. When the eye mask is worn, the left and right reference electrodes respectively contact mastoid processes posterior to junctions of left and right auricles with cheeks to provide a stable, low-amplitude reference potential. As shown in FIGS. 3A-3B, when the eye mask is worn, the eye mask strap 1 encircles the entire head circumference, the left reference electrode 11 and the right reference electrode 14 respectively adhere to points above the left and right auricles, while the left bone conduction earphone 12 and the right bone conduction earphone 13 respectively adhere to points over preauricular temporal regions.

it is worth noting that the present disclosure optimizes the placement of reference electrodes. The reference electrodes are respectively disposed at mastoid processes posterior to junctions of left and right auricles with cheeks. Compared to the occipital region, the area adjacent to the mastoid region is less affected by head movement and free from complex external signal interference (e.g., ECG signals). Compared to the forehead and the tip of the nose, the skin adjacent to the mastoid process is thinner and has lower sweat production, ensuring long-term stability of the electrode-skin interface. Meanwhile, the mastoid process behind the ear is a bony protrusion with a thicker bone structure that can isolate signals from brain activity. Furthermore, the mastoid process is located in the middle and rear of the entire skull, far from the main electrical activity areas of the cerebral cortex, enabling the provision of a reference potential unaffected by brain activity as the reference signal.

It is worth noting that, in a preferred solution, the present disclosure designs specific electrophysiological monitoring electrode placement according to the anatomical features of Asians, and uses flexible hook-and-loop fasteners to achieve adjustable electrode placement, ensuring that positions do not require resetting after initial setup and remain consistently correct. The human forehead length (from the hairline to the upper edge of the eye) is approximately 6-8 cm, the forehead width (from the hairline on both sides to the inner canthi of the eyes) is approximately 10-12 cm, the intercanthal distance (horizontal distance between the inner canthi of the eyes) is approximately 2.5-3.5 cm, and the eye length (horizontal distance from the outer canthus to the inner canthus) is approximately 2.2-3.0 cm. Therefore, based on the facial anatomical features of Asian adults, four 2×2 cm² loop sides are disposed on the eye mask liner 21 to place corresponding electrodes, at points 4-6 cm left and right of a midline and 0.5-2.5 cm from the upper edge, and at points 6-8 cm left and right, 0.5-2.5 cm from the lower edge, and 4-6 cm from the upper edge. Various electrophysiological monitoring electrodes adhere to the eye mask liner 21 via the hook-and-loop fasteners and are electrically connected to the internal circuit 25 through wires threaded through the eye mask liner 21.

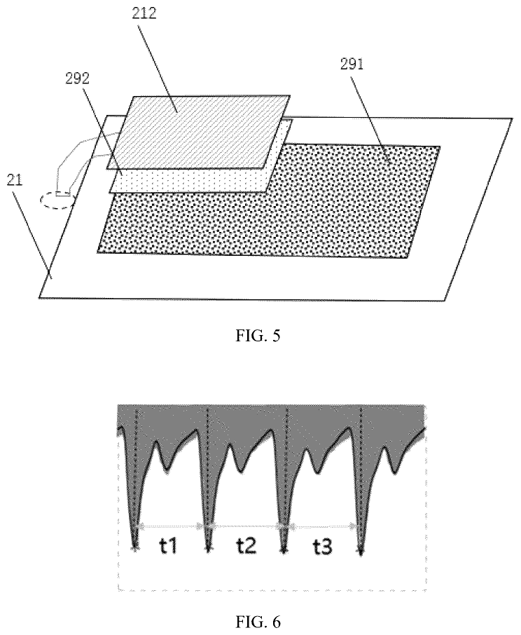

As shown in FIG. 5, the hook-and-loop fastener includes loop side 291 and hook side 292. Each electrode is 1×1 cm² in size, each loop side 291 is 2×2 cm² in size, and each hook side 292 is the same size as the electrode, being 1×1 cm². The loop side 291 adheres to the eye mask liner 21 via adhesive backing. The electrode adheres to the hook side 292 via adhesive backing. The loop side 291 and the hook side 292 are used in conjunction to achieve adjustable electrode placement and fixation. During initial wear, the user may adjust the electrodes according to their facial structure and instructions and adhere the electrodes to the corresponding positions on the loop side 291 via the hook side 292. Thus, no adjustment is required for subsequent wear.

In a preferred solution, to ensure good contact between various types of electrodes and the skin, the electrodes (EEG, EOG, and reference electrodes) need to be flexible, able to deform and adapt to different worn states like the fabric of the eye mask. Additionally, the eye mask strap 1 needs to be repeatedly stretched during wear. Therefore, the reference electrodes and connecting wires need sufficient stretchability to avoid breakage during strap stretching, thereby ensuring stable signal acquisition and transmission. As shown in FIG. 4A, these electrodes each can be prepared based on an ultra-thin metal foil patterned with a serpentine trace and formed via high-precision laser cutting. Compared to rigid electrodes like silver/silver chloride or gold cup electrodes, flexible electrodes offer higher skin conformability and comfort, effectively avoiding poor contact due to uneven skin. As shown in FIG. 4B, the wires between each electrode and the internal circuit can also adopt serpentine routing to effectively prevent circuit breakage caused by eye mask stretching and deformation. Furthermore, an ultra-thin and stretchable polyimide (PI) film can be used for insulating encapsulation of the serpentine routing via methods like hot melting and hot pressing, ensuring stable and reliable signal transmission.

Based on this, various electrophysiological monitoring electrodes can be made of gold foil, with the main fabrication steps as follows. First, a specific serpentine pattern is designed using computer-aid design (CAD) software to enhance electrode flexibility. The gold foil is placed on a polydimethylsiloxane (PDMS) substrate for laser cutting (e.g., 80% power for 40 passes). The gold foil at an edge is removed using tweezers. To prevent deformation of the serpentine pattern, the remaining portion is adhesively lifted with a polyurethane (PU) film. The resulting electrode is placed on PU reactive hot-melt adhesive for high-temperature press forming. The formed electrode is soldered to one end of a wire, and the other end of the wire is connected to the internal circuit 25.

The reference electrode is mainly made of a PU material, Ag/AgCl paste, and copper foil, with the fabrication steps as follows. First, the copper foil is cut to have a width substantially the same as the strap width and a length of approximately 5 mm. The copper foil adheres to a non-adhesive side of the PU material via a double-sided tape. The Ag/AgCl paste is applied to the smooth and wrinkle-free non-adhesive side of the PU material, covering ½ area of the copper foil, and is placed in an oven at 60° C. for 1 hour. The dried PU material coated with Ag/AgCl is cut to a width substantially the same as the width of the eye mask strap. The adhesive side adheres to the elastic band 15, one end of a wire is soldered to the copper foil, and the other end of the wire is connected to the internal circuit 25.

The semiconductor cooling module 26 is embedded in the support sponge 23. The semiconductor cooling module may be in sheet form, but in other embodiments, it may be configured in other structural forms according to the structural characteristics of the eye mask, which is not limited herein. The semiconductor cooling module 26 operates based on a thermoelectric effect, primarily transferring heat from one side to the other through the thermoelectric effect of a thermoelectric material, thereby achieving cooling. Therefore, the semiconductor cooling module 26 includes a front side and a back side. The front side contacts the support sponge 23 and cools the forehead of the human body, while the back side closely adheres to the eye mask shell 22 and dissipates heat through the eye mask shell 22. The semiconductor cooling module 26 is connected to the internal circuit 25 via a wire.

The PPG sensor 27 and the temperature sensor 28 are also disposed on the inner side of the eye mask liner 21. The PPG sensor 27 and the temperature sensor 28 are embedded in an upper left portion of the eye mask liner 21 and are electrically connected to the internal circuit 25 via wires threaded through the eye mask liner 21. When the smart eye mask is worn, the PPG sensor 27 and the temperature sensor 28 adhere to points adjacent to the left supraorbital artery of the human body.

The PPG sensor 27 includes a light source and a signal receiver. It acquires a PPG signal (also referred to as “pulse wave signal”) from the human body by emitting and receiving red and infrared (IR) light, and converts the PPG signal into a digital signal for output. For example, a common commercial MAX30102 sensor may be selected. Based on the PPG signal output by the PPG sensor 27, the HR, HRV, and SpO₂ data of the human body are calculated. Specific calculations may be performed in the internal circuit 25, and after calculation, the data is transmitted to a host computer. Alternatively, the calculations may be performed on the host computer, and the acquired PPG signal is transmitted directly to the host computer.

In an optional solution, for example, the HR is calculated via a beat-to-beat SpO₂ calculation method. The HR and HRV are calculated primarily based on an IR light signal in the PPG signal. The HR HR is determined by calculating a mean RR interval (the time interval between the R waves of two consecutive heartbeats):

$$HR = \frac{60}{RR interval} \qquad ( 1 )$$

where 60 denotes 60 seconds, and the unit of the RR interval is seconds.

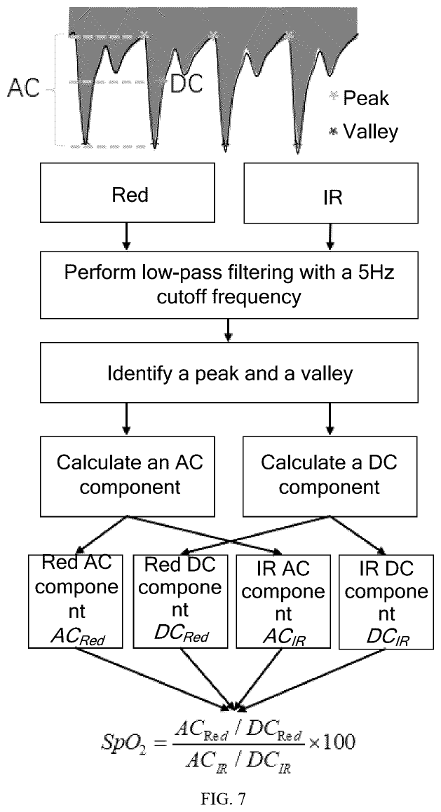

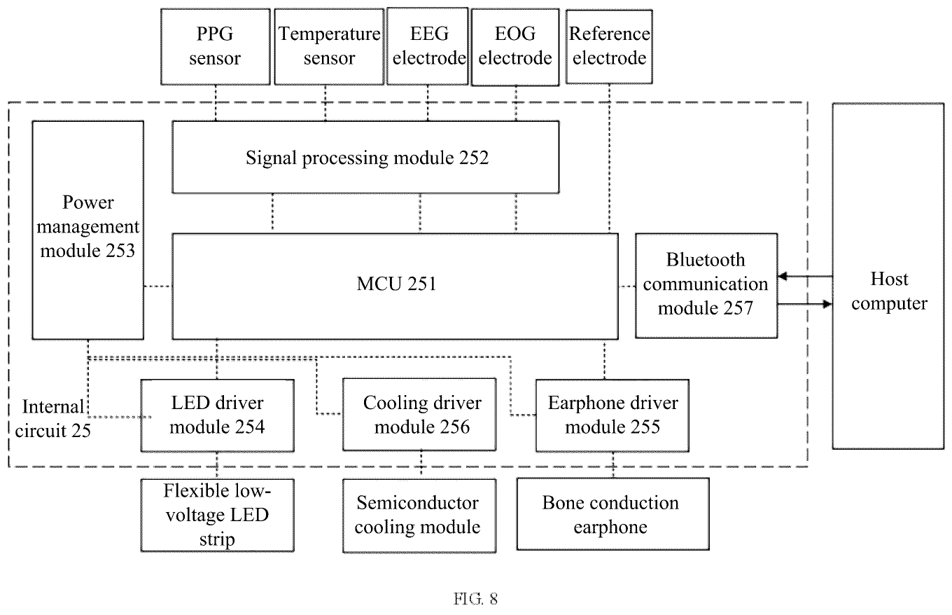

As shown in FIG. 6, in another optional solution, for example, the HR is calculated via a time window method. The specific HR HR calculation process includes setting time window ΔT of a specific time (taking 5 seconds as an example). Within this time window, a newly acquired 1-second pulse wave is stored every second, and the pulse wave of the earliest 1 second of the 5 seconds is removed. The main valley of the pulse wave within this data segment is identified, and its corresponding time is recorded. The mean RR interval is calculated based on multiple peak times, and then the HR is calculated as the HR value for that second:

$$HR = \frac{60}{mean ( {\sum }_{i = 1}^{i = N} {t}_{i + 1} - {t}_{i} )} \qquad ( 2 )$$

where i denotes a pulse wave sequence number within the time window, tᵢ denotes the time of an i-th pulse wave valley, tᵢ₊₁ denotes the time of an (i+1)-th pulse wave valley, and N denotes a total number of pulse waves within the time window, which is 3 in this embodiment.

In an optional solution, based on the pulse wave signal acquired by the PPG sensor 27, taking the beat-to-beat SpO₂ calculation method as an example, the flowchart of SpO₂ data calculation is shown in FIG. 7. The red and IR light in the pulse wave signal first undergo 5 Hz low-pass filtering, then the peaks and valleys of these two signal waveforms are identified. The absolute value of the difference between the peak and valley of a single beat is taken as an alternating current (AC) value for that beat, and the mean value of all data points between two consecutive peaks is taken as a direct current (DC) value for that beat. The SpO₂ for that beat is calculated according to Eq. (3):

$${Sp O}_{2} = \frac{( {AC}_{Red} / {DC}_{Red} )}{( {AC}_{IR} / {DC}_{IR} )} \times 100 \qquad ( 3 )$$

where SpO₂ denotes the blood oxygen saturation; AC_{Red} denotes the AC component of the red light; DC_{Red} denotes the AC component of the IR light; AC_{IR} denotes the DC component of the red light; and DC_{IR} denotes the DC component of the IR light.

In another optional solution, to increase the sampling rate of the SpO₂ signal, a time-window-based method can be used to calculate SpO₂ once per second, ensuring a stable sampling rate (1 Hz), and this approach decouples the acquisition speed of SpO₂ data from the subject's heart rate (HR). Specifically, time window ΔT of specific time is first set (taking 5 seconds as an example). Within this time window, newly acquired pulse waves are stored every second, and the earliest 1-second pulse wave is removed. The main wave peak and valley of the pulse wave are identified from the time window data. The average of the absolute values of differences between the peak and valley of each beat is taken as the AC value for this time window, and the mean of all data points is taken as the DC value for the beats within this time window. The SpO₂ is calculated according to Eq. (3).

The temperature sensor 28 is mainly configured to monitor the core temperature of the human body in real time. It can provide circadian rhythm information to users by monitoring overnight body temperature. The temperature sensor 28 is connected to the internal circuit 25 via a wire. The iontronic temperature sensor features high sensitivity, high selectivity, and biomimetic properties, capable of detecting temperature differences smaller than 10 millikelvins. Their response to temperature changes is independent of mechanical deformation, making them suitable for wearable devices and flexible electronics applications. Therefore, the sensor can serve as a preferred solution. Understandably, in other embodiments, temperature sensors based on other principles such as capacitive or resistive can also be adopted, and the present disclosure does not impose limitations on this.

As shown in FIG. 7, the internal circuit 25 mainly includes microcontroller unit (MCU) 251 and functional modules electrically connected to the MCU 251, such as signal processing module 252, power management module 253, LED driver module 254, earphone driver module 255, and Bluetooth communication module 257. The EEG electrodes, the EOG electrodes, the PPG sensor 27, and the temperature sensor 28 are electrically connected to the MCU 251 through the signal processing module 252. The reference electrode is directly connected to a reference electrode port of the MCU 251 via a wire. The signal processing module 252 is configured to perform amplification, digital-to-analog conversion, and analog-to-digital conversion on an input signal. For example, it can use an ADS1299 chip. The signals acquired by the EEG electrodes and the EOG electrodes are analog signals and are digitized into transmittable digital signals through the ADS1299 chip. Since the pulse wave signal sent by the PPG sensor 27 is a digital signal, the signal processing module 252 provides a dedicated digital-to-analog converter (DAC) circuit for the PPG sensor 27 to enable data transmission between the PPG sensor 27 and the MCU 251. The DAC circuit is configured to convert the pulse wave signal acquired by the PPG sensor 27 into an analog voltage and input it to the ADS1299 chip for digitization, thereby achieving unified digitization of all signals. The power management module 253 can adopt a 3.7 V to 1,000 mAh lithium battery, which is connected to the MCU 251, the LED driver module 254, the earphone driver module 255, and the cooling driver module 256 via wires, ensuring all-night signal acquisition and management. The LED driver module 254, the earphone driver module 255, and the cooling driver module 256 are electrically connected to the LED light strip 24, the bone conduction earphones, and the semiconductor cooling module 26, respectively. The Bluetooth communication module 257 is communicatively connected to the host computer. In a preferred solution, the internal circuit 25 may be designed based on a flexible printed circuit (FPC) board to better adapt to and be disposed inside the eye mask body 2.

The bone conduction earphones may directly adopt commercial bone conduction earphones. The bone conduction earphones are connected to the MCU 251 of the internal circuit 25 through the earphone driver module 255. When the earphones operate, the MCU 251 first enables the earphone driver module 255 and sends an electrical signal carrying audio information to the earphone driver module 255. The earphone driver module 255 converts the electrical signal into a vibration signal, which is ultimately converted into audio by the human auditory system.

The eye mask shell 22 has an overall length of 18 cm and a width of 8 cm, and is internally provided with a recess for holding the internal circuit 25 and the LED light strip 24. The internal circuit 25 and the LED light strip 24 are disposed in the recess. The LED light strip 24 is powered by either 5 V DC power supply or a 5 V pulse width modulation (PWM) wave to adjust brightness. The LED light strip has a density of 60 LEDs/m. The LED light strip is bent and adhered to the interior of the eye mask shell 22 with glue or other adhesive substances, such that it corresponds to an eye recess of the support sponge 23 and is covered by the eye mask liner 21. The LED light strip 24 is connected to the internal circuit 25 via a wire and controlled by the internal circuit 25, emitting 40 Hz monochromatic light with a sleep-inducing effect. Optionally, an edge of the eye mask shell 22 may include functional buttons such as device power button 221, music play/pause button 222, LED light strip on/off button 223, brightness up button 224, brightness down button 225, and cooling on/off button 226.

Furthermore, Embodiment 2 of the present disclosure provides a wearable non-intrusive smart eye mask system. The system includes the smart eye mask described in Embodiment 1 and a host computer communicatively connected to the smart eye mask. The host computer may be an electronic device such as a smartphone, a tablet computer, a dedicated terminal device, a personal computer (PC), a smartwatch, or a smart bracelet. The electronic device typically includes a processor, a memory, a communication module, and a human-computer interaction interface. The processor is mainly configured to perform sleep monitoring, and the memory stores music and acquired physiological data. The communication module is mainly configured to receive data acquired by the smart eye mask and to send relevant commands generated by the processor (including enabling or disabling sleep-inducing audio/sleep-inducing light, etc.) to the smart eye mask, and has Bluetooth communication capability. The human-computer interaction interface includes thermal regulation option buttons such as start signal acquisition, light on/off, music on/off, and system presets. The user clicks the buttons to achieve corresponding functions. The processor is configured to receive data such as EEG signals, EOG signals, HR, and SpO₂ sent by the smart eye mask, perform sleep staging, determine the duration of each stage, and control the LED light strip 24, the bone conduction earphones, and the semiconductor cooling module 26 for sleep intervention. Understandably, there is one or more computer programs stored in the memory of the host computer, and the computer program is executed to implement the following operations. The HR and the SpO₂ are calculated based on the PPG signal; sleep monitoring is performed in real time based on the EEG signal, the EOG signal, the HR, and the SpO₂; and the intervention component is controlled to perform sleep intervention when a sleep monitoring result meets a preset condition. The sleep monitoring includes sleep staging and current sleep stage duration calculation. Correspondingly, the sleep monitoring result includes the current sleep stage and its duration. The sleep stage includes a W stage, an N1 stage, an N2 stage, an N3 stage, and an REM stage. Therefore, the present disclosure can accurately distinguish each sleep stage.

Furthermore, Embodiment 3 of the present disclosure provides a sleep monitoring method applicable to the wearable non-intrusive smart eye mask system described in Embodiment 2. The sleep monitoring method mainly includes three phases: a preprocessing phase, a sleep staging phase, and a sleep time (ST) calculation phase. According to the international sleep staging standard, a 30-second ST is selected as an epoch for classification. The sampling rate of the EEG signal, the EOG signal, and the core body temperature signal is set to 100 Hz, and the sampling rate of the PPG signal is set to 40 Hz. In the preprocessing phase, the host computer first applies 0.3-35 Hz bandpass filtering to the EEG signal uploaded by the smart eye mask and 0-15 Hz low-pass filtering to the EOG signal, followed by normalization. The PPG signal uploaded by the smart eye mask undergoes 0-5 Hz low-pass filtering. The HR and the SpO₂ are calculated according to a preset algorithm. Understandably, if the HR and the SpO₂ have already been calculated by the smart eye mask, the calculated HR and the SpO₂ data are directly uploaded to the host computer.

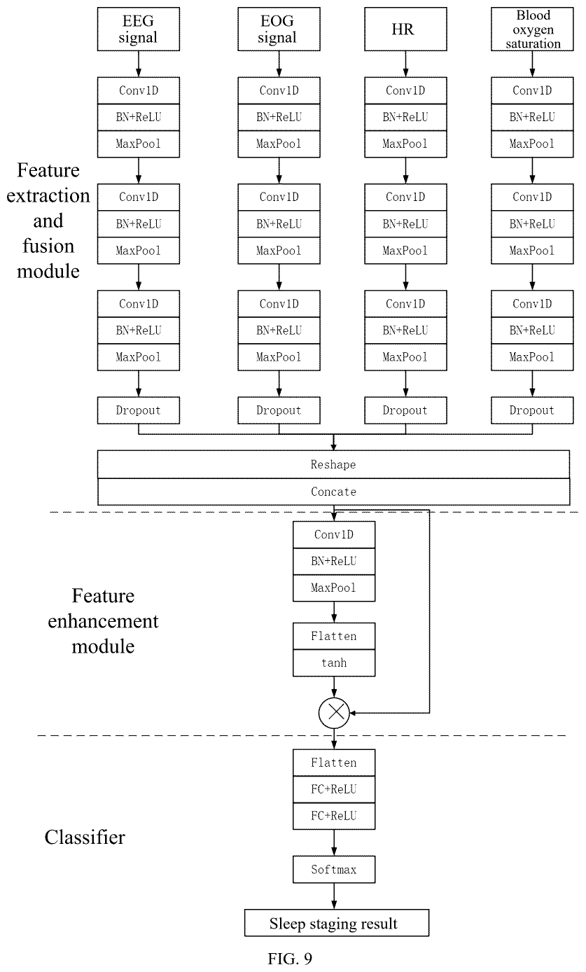

In the sleep staging phase, the host computer uses a pre-trained deep learning algorithm to achieve accurate and real-time sleep staging. As shown in FIG. 9, the network architecture of a sleep staging model based on the deep learning algorithm is mainly divided into three parts. In the first part, the feature extraction and fusion module includes four feature extraction branches configured to extract features from the four signals, i.e. the EEG signal, the EOG signal, the HR, and the SpO₂, and fuse these four types of signals. In the second part, a feature enhancement module performs operations such as amplification and filtering on the fused feature. In the third part, a classifier predicts a sleep stage through the feature.

The four feature extraction branches of the feature extraction and fusion module each mainly include serially connected convolutional layers, a Dropout layer, a Reshape layer, and a Concate layer. In each branch, the signal passes through three serially connected convolutional layers. In each convolutional layer, a high-dimensional feature value is first calculated through a one-dimensional convolution operation (Conv1D), and batch normalization (BN) transforms the feature value into a standard normal distribution with a mean of 0 and a variance of 1. Next, a rectified linear unit (ReLU) activation function is applied for nonlinearization, and max pooling (MaxPool) extracts a local maximum value of the feature to reduce the feature size. Finally, the Dropout layer randomly discards a portion of the feature value to prevent overfitting. Due to differences in sizes of the input signal and parameters of the convolutional layers, the feature sizes extracted by the four feature extraction branches also differ. The Reshape layer maps features extracted by different feature extraction branches to the same size, and the Concate layer combines the features into feature array Feature1.

The feature enhancement module mainly includes a convolutional layer, a flatten layer (Flatten), and a hyperbolic tangent (tanh) activation function. The feature array Feature1 passes through the convolutional layer in the feature enhancement module. The convolutional layer includes a one-dimensional convolution operation (Conv1D), further increasing the feature dimension of Feature1. After convolution, Feature1 is reduced to a one-dimensional array via Flatten. The tanh activation function scales the values of the one-dimensional array to the range of −1 to 1, thereby obtaining the weight value for each feature. The feature array Feature1 is multiplied by the weight values to obtain enhanced feature Feature2, achieving amplification and filtering of different features and reflecting differences in importance between features. Finally, the enhanced feature Feature2 is input to the classifier in the third part.

The classifier mainly includes a flatten layer (Flatten), two serially connected fully connected (FC) layers with ReLU activation functions (FC+ReLU), and a Softmax layer. The enhanced feature Feature2 is processed by Flatten to convert the multidimensional array into a one-dimensional array. The one-dimensional array sequentially passes through the two fully connected layers with ReLU activation functions to map the feature array to a classification array, i.e., an array of size (1,5). The Softmax layer scales the values of the classification array to 0-1, representing the classification probabilities of the five sleep stages (including the W stage, N1 stage, N2 stage, N3 stage, and REM stage). The sleep stage corresponding to the highest probability is the predicted result of sleep staging. The above sleep staging method uses convolution operations, featuring high speed and efficiency, and can accurately output sleep staging monitoring results in real time.

Based on the above analysis, during the sleep staging phase, the present disclosure uses four input signals (EEG, EOG, HR, and SpO₂ signals) for sleep staging, reducing various interferences that a single signal may suffer (such as improper electrode placement and noise), greatly decreasing staging errors. In other words, the multi-modal signals can complement each other, reducing the impact of errors or noise in any single signal on the entire staging system, thereby improving the robustness and accuracy of sleep staging.

It is worth noting that in the present disclosure, the network architecture of the sleep staging model based on the deep learning algorithm involves multi-modal feature extraction, fusion, enhancement, and an efficient classifier, achieving high-precision, real-time sleep staging. In the sleep staging model, the feature extraction and fusion module uses four independent feature extraction branches to extract features from the EEG, EOG, HR, and SpO₂ signals, optimizing feature expression through convolution operation (Conv1D), BN, ReLU activation function application, and max pooling (MaxPool), and combining Dropout to prevent overfitting. The feature enhancement module dynamically assigns feature weights through the convolutional layer and the tanh activation function, amplifying key features (such as slow-wave activity in the N3 stage) and filtering redundant information. The classifier uses the fully connected layers (FC) and Softmax layer to map features to the five sleep stages, outputting a probability distribution. Particularly, the efficiency and parallel computing capability of convolution operations enable the model to process data in real time, meeting the low-latency requirements of wearable devices. The complementarity of the multi-modal signals (such as slow waves on EEG and HRV) and the feature enhancement mechanism significantly improve the recognition accuracy of the N3 stage. Meanwhile, Dropout and BN enhance the generalization ability of the model, making the model adapt to individual differences and noise interference.

In the ST calculation phase, the duration of the current sleep stage is calculated in real time based on sleep staging result. Specifically, if the sleep stage remains unchanged, the duration of the sleep stage increases by one sleep epoch (30s), while the durations of other sleep stages are zero. If the sleep stage changes, the duration of the previous sleep stage is reset to zero, and the duration of the current sleep stage is set to one sleep epoch. For example, if the current sleep stage is a W stage and lasts 150s but the monitoring result shows N1 stage, the state is modified to N1 with a duration of 30 s. Conversely, if the prediction result is still W stage, the state remains W stage with a duration of 180 s. By calculating the durations of specific N3 stage and W stage, the sleep intervention time is controlled.

Additionally, to assess whether the sleep quality in a certain stage is improved, the duration of the current sleep stage and the total duration of all stages are calculated. In an optional embodiment, first, in the initialized state, a dictionary is created to record the cumulative time of each sleep stage (W, N1, N2, N3, REM). Initially, the durations of all stages are zero. Meanwhile, the current sleep stage and its duration are defined. Each time a new sleep stage monitoring result is generated, processing is performed based on whether the stage changes. If the current stage is the same as the previous stage, the duration of this stage increases by one sleep epoch (30 seconds). If the current stage differs from the previous stage, the duration of the previous stage is recorded in the corresponding total duration, the duration of the current stage is reset to a new epoch time (30 seconds), and the current sleep stage is updated to the new stage. At each stage change, the current stage duration is promptly accumulated into the total duration of the corresponding stage, ensuring accurate final duration for each stage. Finally, at the end of the entire sleep epoch (or at the end of a stage change), the total duration of each sleep stage is returned. That is, the total duration of each stage is the cumulative duration from sleep onset to sleep end, in seconds. The calculated duration data of each sleep stage over multiple days are acquired to analyze the wearer's sleep structural characteristics, assess sleep quality and improvements, and further serve as data for insomnia diagnosis and treatment.

Given known sleep stages, doctors can determine a patient's sleep structure and determine whether the patient suffers from insomnia based on the sleep state of the patient, and intervene in and treat insomnia if necessary. Existing insomnia intervention methods include medication and many physical methods without side effects. Current physical methods include light stimulation, acoustical stimulation, transcranial electrical stimulation, thermal stimulation, transcranial magnetic stimulation, etc. Transcranial magnetic stimulation solutions cannot be miniaturized, and transcranial electrical stimulation requires more complex designs. 40 Hz light stimulation and acoustical stimulation have clear mechanisms and are easy to implement in wearable designs, making them optimal solutions selectable for closed-loop sleep management. Additionally, some current acoustical stimulation solutions play synchronized pink noise pulses during slow-wave sleep, which can significantly increase the time and quality of deep sleep. Forehead cooling stimulation has also been proven to promote subjects entering sleep states and increase the ST. Based on this, the present disclosure can organically integrate sleep monitoring with sleep intervention, perform targeted interventions based on the sleep stage, form closed-loop sleep management, and allow personalized settings according to needs.

Furthermore, Embodiment 4 of the present disclosure provides a sleep intervention method based on the sleep staging result of Embodiment 3. When the user wears the wearable non-intrusive smart eye mask system for the first time, the initial positions of each electrode are determined according to the standard, and the electrode positions are adjustable through hook-and-loop fasteners. After proper wearing, the user can first preset functions on the host computer, including whether to enable pre-sleep sleep-inducing audio and/or light exposure and/or thermal regulation interventions, whether to enable deep sleep regulation, and audio selection, etc. Understandably, after the above settings are completed, parameters can be adjusted on the host computer as needed subsequently.

Typically, the intervention time for the sleep-inducing audio, light, and thermal regulation is 10-30 minutes, ensuring the intervention is effective without disturbing the normal sleep cycle. Whether to perform deep sleep regulation can also be flexibly determined according to user needs, with the deep sleep regulation time determined by the user's actual deep ST. After the user selects to enable deep sleep regulation, the host computer sends relevant commands to the smart eye mask via the communication module. The relevant commands are received by the Bluetooth communication module 257 in the internal circuit 25 and transmitted to the MCU 251. The MCU 251 modifies internal logic to execute regulation of the light PWM wave, regulation of the cooling driver module PWM wave, and regulation of the earphone driver module 255. Furthermore, in the initial state, it can be defaulted to automatically enable the sleep-inducing mode when the user meets insomnia criteria, and automatically enable the deep sleep regulation mode after the user enters the deep sleep stage.

To use the wearable non-intrusive smart eye mask system, the user presses the device power button 221 on the smart eye mask, such that the system starts running. First, the host computer and the user predetermine intervention information, including sleep-inducing intervention method selection, whether to enable deep sleep regulation, sleep-inducing and deep sleep audio selection, etc. After intervention information is determined, the MCU 251 starts signal acquisition and data transmission, and the host computer begins real-time sleep staging. When the system determines through sleep monitoring that the current stage is W stage (persistent wakefulness) and calculates that the user's W stage lasts over 20 minutes, it is determined that the user meets the insomnia criteria. At this point, the system automatically triggers the selected sleep-inducing intervention method according to preset intervention information to help the user fall asleep faster. The intervention methods mainly include 40 Hz flashing light (achieved by the internal circuit 25 controlling the LED light strip 24), forehead cooling (achieved by the internal circuit 25 controlling the semiconductor cooling module 26), and sleep-inducing audio (achieved by the internal circuit 25 controlling the bone conduction earphone 12 and bone conduction earphone 13). The 40 Hz flashing light simulates light pulses at specific frequencies, promoting sleep onset by modulating neural activity. The sleep-inducing audio includes white noise, natural sounds (such as rain, ocean waves), meditation music, autonomous sensory meridian response (ASMR), etc., to relax the body and mind and promote sleep onset. The semiconductor cooling module 26 can combine with the temperature sensor 28 to achieve closed-loop body temperature detection and control.

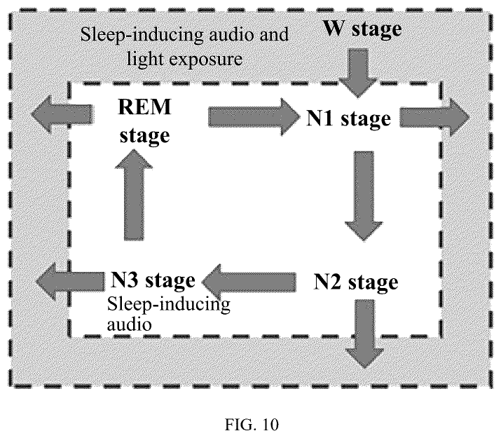

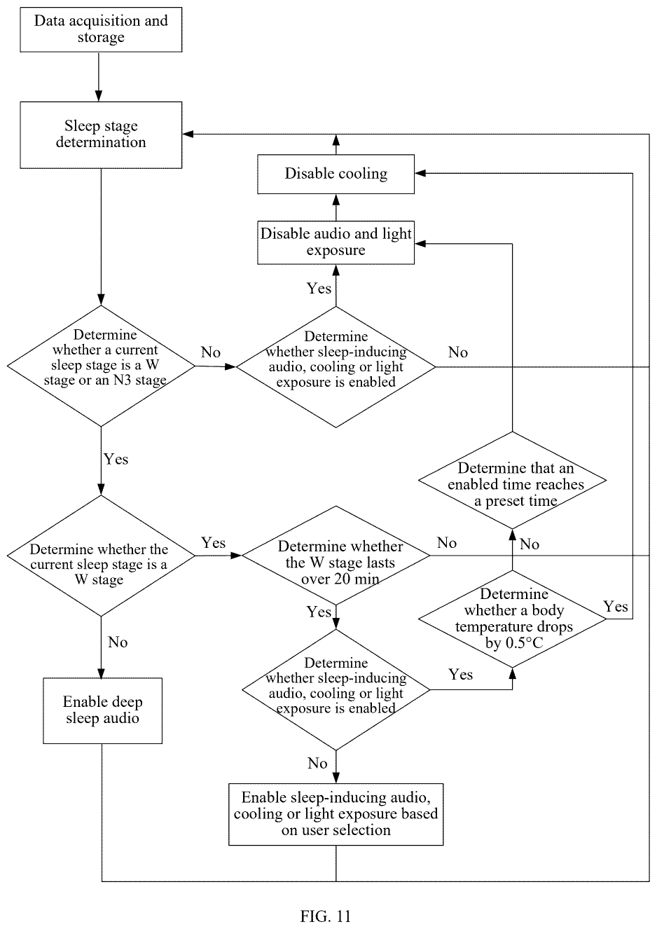

As shown in FIG. 10 and FIG. 11, when the host computer monitors that the current stage is W stage (persistent wakefulness) and detects that the user's W stage lasts over 20 minutes, it sends a command to start sleep-inducing intervention to the smart eye mask. After the MCU 251 receives the command to start sleep-inducing intervention, it first determines whether to enable light, cooling, or sleep-inducing audio based on preset intervention methods. If light exposure needs to be enabled, the PWM output is enabled, and the MCU 251 sends a 40 Hz PWM wave. After passing through the LED driver module 254, the current and voltage of the PWM wave are amplified to the level required to drive the LED light strip 24. During light exposure, the user can press the brightness up button 224 or brightness down button 225 on the shell to adjust the duty cycle of the PWM wave. When the brightness needs to be increased, the duty cycle of the PWM wave is increased. When brightness needs to be decreased, the duty cycle of the PWM wave is decreased. When the user presses the LED light strip on/off button 223, if the current duty cycle is 0, it indicates that the LED light strip 24 is currently off, and the duty cycle is set to 30% to turn on the LED light strip. If the current duty cycle is not 0, it indicates that the LED light strip is currently on, and the duty cycle is set to 0% to turn off the LED light strip 24. If light exposure is not enabled, the PWM output is disabled.

Similarly, to enable the sleep-inducing audio, the earphone driver module 254 is enabled, and an electrical signal carrying audio information is sent to the earphone driver module 254. The earphone driver module 254 amplifies the current and voltage of the electrical signal carrying audio information to the level required to drive the bone conduction earphones. If it is not necessary to enable the sleep-inducing audio, the earphone driver module 254 is disabled.

It should be noted that if it is necessary to enable the semiconductor cooling module 26, the PWM output is enabled. The MCU sends a PWM wave of a specific frequency, which controls the semiconductor cooling module 26 via the cooling driver module 256. A larger duty cycle of the PWM wave indicates a stronger cooling effect. Considering user experience and sleep impact, the semiconductor cooling module 26 is used in conjunction with the temperature sensor 28. After the PWM wave is enabled for the semiconductor cooling module 26, the temperature sensor 28 continuously acquires body temperature signals. If it is detected that the user's forehead temperature drops by more than 0.5° C., even if the user has not entered sleep state, the MCU 251 disables the PWM output of the cooling driver module 256 in advance to end cooling.

According to user selection, when the intervention time exceeds the user-set time, all intervention methods are disabled. When the host computer detects that the user enters a sleep state (the sleep staging result is a non-W stage that lasts 3 minutes), it sends a command to stop sleep-inducing intervention to the smart eye mask. After the MCU 251 in the smart eye mask receives the command, it disables the PWM output and the earphone driver module, stopping sleep-inducing. Research shows that pink noise can help consolidate the deep sleep stage and improve sleep quality. Specifically, the audio of pink noise can be designed as 50 ms pulses. Each pulse has a 5 ms rising edge and falling edge to avoid adverse stimulation to sleep while promoting slow-wave activity and extending deep ST. Therefore, based on the precise sleep staging in Embodiment 3, sleep intervention can be achieved for specific deep sleep stages.

In an optional solution, deep sleep intervention is incorporated. If the preset intervention information selects to enable the deep sleep regulation mode, the system automatically activates a dedicated audio intervention mechanism after monitoring that the user enters an N3 stage (deep sleep stage). Specifically, the host computer first sends a command to start deep sleep intervention to the smart eye mask. After receiving the command, the MCU 251 in the smart eye mask enables the earphone driver module 254 and simultaneously sends an electrical signal carrying pink noise audio information to the earphone driver module 255. The earphone driver module 255 converts the electrical signal into a vibration signal, which is ultimately converted into specific deep sleep extension audio (pink noise) by the human auditory system. This process is played at intervals without interfering with natural sleep rhythms. When it is monitored that the N3 stage ends (usually changing to an N2 stage), the host computer sends a command to stop deep sleep intervention. After receiving the command, the MCU 251 disables the PWM output, thereby stopping deep sleep intervention.

Finally, it should be noted that although the implementations of the present disclosure are described above with reference to the drawings, the present disclosure is not limited to the aforementioned specific implementations and application fields, and the above specific implementations are merely illustrative and instructive, rather than restrictive. Those of ordinary skills in the art can make various forms under the inspiration of this specification and without departing from the protection scope of the claims of the present disclosure, and these forms all fall within the protection scope of the present disclosure.

1. A closed-loop sleep management method, applied to a head-worn closed-loop sleep management device, and comprising:
performing sleep monitoring in real time based on an electroencephalogram (EEG) signal, an electrooculogram (EOG) signal, a heart rate (HR), and a blood oxygen saturation (SpO₂); and
controlling an intervention component to perform sleep intervention when a sleep monitoring result meets a preset condition;
wherein the sleep monitoring comprises sleep staging and calculation of a duration of a current sleep stage; the sleep monitoring result comprises the current sleep stage and the duration thereof; and a sleep stage comprises a wake (W) stage, an N1 stage, an N2 stage, an N3 stage, and a rapid eye movement (REM) stage.

2. The closed-loop sleep management method according to claim 1, further comprising:
acquiring the EEG signal, the EOG signal, and a photoplethysmography (PPG) signal; and
calculating the HR and the SpO₂ based on the PPG signal.

3. The closed-loop sleep management method according to claim 1, wherein the sleep staging is implemented by a sleep staging convolutional neural network (CNN); and the sleep staging CNN comprises:
a feature extraction and fusion module, configured to: extract four types of signal features from the EEG signal, the EOG signal, the HR, and the SpO₂ respectively through four feature extraction branches, and fuse the four types of signal features to obtain a fused feature array, wherein the four feature extraction branches each optimize feature expression through a first convolutional layer, batch normalization (BN), a rectified linear unit (ReLU) activation function, and max pooling (MaxPool), and incorporate Dropout to prevent overfitting;
a feature enhancement module, configured to: dynamically assign a feature weight to the fused feature array through a second convolutional layer and a hyperbolic tangent (tanh) activation function, amplify a key feature, and filter redundant information; and
a classifier, configured to: map, through a fully connected (FC) layer and a Softmax layer, an enhanced feature array to the W stage, the N1 stage, the N2 stage, the N3 stage, and the REM stage.

4. The closed-loop sleep management method according to claim 1, wherein when the preset condition is met, the sleep intervention comprises:
performing sleep-inducing intervention when the current sleep stage is the W stage and a calculated duration of the W stage exceeds a first preset time; and stopping the sleep-inducing intervention when the current sleep stage changes from the W stage to a non-W sleep stage and a calculated duration of the non-W sleep stage exceeds a second preset time.

5. The closed-loop sleep management method according to claim 1, wherein when the preset condition is met, the sleep intervention further comprises:
performing deep sleep intervention when the current sleep stage is the N3 stage; and stopping a command of the deep sleep intervention when the current sleep stage changes from the N3 stage to another sleep stage.

6. The closed-loop sleep management method according to claim 1, wherein the sleep intervention comprises at least one of lighting intervention, audio intervention, and forehead cooling intervention.

7. The closed-loop sleep management method according to claim 1, wherein the sleep monitoring further comprises calculating a total duration of each of the W stage, the N1 stage, the N2 stage, the N3 stage and the REM stage.

8. A head-worn closed-loop sleep management system, comprising: a head-worn closed-loop sleep management device and a computer program, wherein
the head-worn closed-loop sleep management device comprises a head-worn wearable mechanism, a signal acquisition component, an internal circuit and an intervention component, wherein the signal acquisition component, the internal circuit and the intervention component are disposed on the head-worn wearable mechanism;
the signal acquisition component comprises an EOG electrode, an EEG electrode, a reference electrode, and a PPG sensor; the EOG electrode is configured to acquire an EOG signal; the EEG electrode cooperates with the reference electrode to acquire an EEG signal; and the PPG sensor is configured to acquire a PPG signal from a human body;
the internal circuit is configured to preprocess the EOG signal, the EEG signal and the PPG signal acquired by the signal acquisition component, and control the intervention component to perform sleep intervention according to a received intervention command;
the computer program is stored in the internal circuit and/or an electronic device communicatively connected to the head-worn closed-loop sleep management device; and
the computer program is executed to implement the closed-loop sleep management method according to claim 1.

9. The head-worn closed-loop sleep management system according to claim 8, wherein a pair of EOG electrodes are respectively positioned below an outer canthus of a left eye of the human body and above an outer canthus of a right eye of the human body when the head-worn wearable mechanism is worn;
a pair of EEG electrodes are respectively positioned at left and right sides of a prefrontal region of the human body when the head-worn wearable mechanism is worn; and
a pair of reference electrodes are positioned to respectively contact mastoid processes posterior to junctions of left and right auricles with cheeks of the human body when the head-worn wearable mechanism is worn.

10. The head-worn closed-loop sleep management system according to claim 9, wherein when the head-worn wearable mechanism is worn, the pair of EEG electrodes are positioned 4 cm-6 cm left and right respectively from a midline of an inner side of an eye mask and 0.5 cm-2.5 cm from an upper edge of the eye mask, and the pair of EOG electrodes are positioned 6 cm-8 cm left and right respectively from the midline of the inner side of the eye mask, 0.5 cm-2.5 cm from a lower edge of the eye mask, and 4 cm-6 cm from the upper edge of the eye mask.

11. The head-worn closed-loop sleep management system according to claim 9, wherein the pair of EOG electrodes and the pair of EEG electrodes each are detachably connected to the head-worn wearable mechanism through a flexible hook-and-loop fastener; the hook-and-loop fastener comprises a loop side and a hook side; the loop side is fixed to an inner side of the head-worn wearable mechanism and has an area larger than the hook side; and the pair of EOG electrodes and the pair of EEG electrodes each are bonded to the loop side through the hook side.

12. The head-worn closed-loop sleep management system according to claim 8, wherein the internal circuit comprises a microcontroller unit (MCU), a signal processing module, a first communication module, an intervention driver module and a power supply module, wherein the signal processing module, the first communication module, the intervention driver module and the power supply module are electrically connected to the MCU;
the signal processing module is configured to preprocess the EOG signal, the EEG signal and the PPG signal of the human body input to the internal circuit; and the preprocessing comprises signal amplification, digital-to-analog conversion, and analog-to-digital conversion;
the first communication module is configured to communicatively connect the MCU to the electronic device; and
the intervention driver module is electrically connected to the intervention component.

13. The head-worn closed-loop sleep management system according to claim 12, wherein the intervention component comprises at least one of a flashing light module and a bone conduction earphone; and
the intervention driver module comprises at least one of a light driver module electrically connected to the flashing light module and an earphone driver module electrically connected to the bone conduction earphone.

14. The head-worn closed-loop sleep management system according to claim 13, wherein the intervention component further comprises a semiconductor cooling module;
the intervention driver module comprises a cooling driver module electrically connected to the semiconductor cooling module;
the signal acquisition component further comprises a temperature sensor configured to acquire a temperature signal from the human body; and
the computer program is executed to further implement:
controlling, during operation of the semiconductor cooling module, the semiconductor cooling module to stop sleep intervention when the temperature signal acquired from the human body is below a temperature threshold.

15. The head-worn closed-loop sleep management system according to claim 8, wherein the head-worn wearable mechanism is a flexible structure; the internal circuit is a flexible printed circuit (FPC) board; the EOG electrode, the EEG electrode, and the reference electrode are flexible electrodes; the EOG electrode and the EEG electrode each is configured in a stretchable serpentine pattern; and wires for connecting the EOG electrode, the EEG electrode, the reference electrode, the PPG sensor, the intervention component, and the internal circuit each is configured in a stretchable serpentine routing.

16. The head-worn closed-loop sleep management system according to claim 8, wherein the head-worn wearable mechanism is an eye mask; the eye mask comprises an eye mask body and an eye mask strap; the EOG electrode, the EEG electrode, and the PPG sensor are disposed on a skin-facing side of the eye mask body; the internal circuit is disposed inside the eye mask body; and the reference electrode is disposed on the eye mask strap; and
when the intervention component comprises at least one of a flashing light module, a bone conduction earphone and a semiconductor cooling module, the flashing light module and the semiconductor cooling module are disposed inside the eye mask body, and the bone conduction earphone is disposed on the eye mask strap.

17. An electronic device, comprising: a processor, a memory, and a second communication module, wherein the electronic device is communicatively connected to a head-worn closed-loop sleep management device via the second communication module; and the processor is configured to call a computer program stored in the memory to implement the closed-loop sleep management method according to claim 1.

18. The closed-loop sleep management method according to claim 2, wherein the sleep monitoring further comprises calculating a total duration of each of the W stage, the N1 stage, the N2 stage, the N3 stage and the REM stage.

19. The closed-loop sleep management method according to claim 3, wherein the sleep monitoring further comprises calculating a total duration of each of the W stage, the N1 stage, the N2 stage, the N3 stage and the REM stage.

20. The closed-loop sleep management method according to claim 4, wherein the sleep monitoring further comprises calculating a total duration of each of the W stage, the N1 stage, the N2 stage, the N3 stage and the REM stage.
